(19) United States
(12) Patent Application Publication
(10) Pub. No.: US 2004/0161399 A1
(43) Pub. Date: Aug. 19, 2004

(54) Use of nonimmunosuppressive [$g(g)-hydroxy-n-methyl-l-leucine4]cyclosporin derivatives for treating hair loss

(76) Inventors: Sang-Nyun Kim, Daejeon (KR); Ho-Jeong Ahn, Daejeon (KR); Chang-Woo Lee, Daejeon (KR); Min-Ho Lee, Daejeon (KR); Jung-Hun Kim, Daejeon (KR); Jong-Il Kim, Daejeon (KR); Seung-Jin Kim, Seoul (KR); Ho-Song Cho, Daejeon (KR); Heon-Sik Lee, Daejeon (KR); Hyung-Jin Kim, Daejeon (KR)

Correspondence Address: VENABLE, BAETJER, HOWARD AND CIVILETTI, LLP, P.O. BOX 34385, WASHINGTON, DC, 20043-9998, US

(21) Appl. No.: 10/477,267

(22) PCT Filed: May 11, 2002
(86) PCT No.: PCT/KR02/00880

(30) Foreign Application Priority Data
May 17, 2001 (KR) 2001/27022

(51) Int. Cl. A61K 7/06; A61K 7/11; A61K 38/13

(52) U.S. Cl. 424/70.14; 514/11

(57) ABSTRACT

The present invention discloses a hair growth promoting agent comprising a cyclosporin derivative having an excellent hair growth stimulating ability with little immunosuppressive effect as an active ingredient. In accordance with the invention, there is provided a hair growth promoting agent comprising a cyclosporin derivative represented by Chemical Formula 1 below. The derivatives of the invention were prepared by derivatization of cyclosporins at the amino acid residue 4, N-methyl-L-leucine, and hair growth promoting effects thereof were examined. Such a hair growth promoting agent, comprising a cyclosporin derivative as an active ingredient, exhibits an excellent hair growth effect, while it shows very weak immunosuppressive activity, compared to unmodified cyclosporin A.

### TECHNICAL FIELD

The present invention relates to a hair growth promoting agent comprising a [γ-hydroxy-N-methyl-L-leucine⁴] cyclosporin derivative, having an excellent hair restoring effect with non-immunosuppressive activity, as an active ingredient.

### BACKGROUND ART

On average, the human scalp contains about 100,000 to 150,000 hairs. Each hair has three main stages of growth: anagen, catagen and telogen, after which the hair falls out. This hair growth cycle is repetitive and the duration of one cycle is different from other cycles, ranging from approximately 3 to 6 years. Thus, the average adult normally loses about 50 to 100 hairs every day. In general, alopecia refers to a phenomenon wherein duration of the anagen growth phase is shortened and the percentage of hairs in the catagen and telogen phases increases, whereby the number of lost hairs is abnormally increased.

There are many theories to explain loss of hair, including for example, poor blood circulation, excessive functioning of male sex hormone, excessive production and secretion of sebum, deterioration of scalp by peroxides, bacteria, etc., hereditary factors, aging, stress, etc. However, explicit mechanisms have not been revealed. Recently, the population suffering from hair loss is tending to increase, since changing dietary habits and stress imposed on individuals due to modern social environments, etc. have increased. Also, the age of the individuals affected by alopecia is dropping and furthermore, the population of female alopecia sufferers is rising.

One of preparations which are most commonly used for treatment and prevention of alopecia is one that contains minoxidil. There are two hair-regrowth agents which have received approval from the U.S. Food and Drug Administration, and minoxidil is one of those approved hair-regrowth agents. Minoxidil was originally developed as a hypertension drug for the purpose of reducing blood pressure. However, when using this drug, as a side effect, a trichogenous effect was observed and thereafter, this drug became famous as a hair-regrowth agent. Although mechanisms by which minoxidil works as a hair-regrowth agent is not clearly understood, it is inferred that minoxidil increases blood flow by expansion of blood vessels, whereby roots of hairs are supplied with more nutrition and eventually, growth of hairs are promoted.

Such a model of blood flow increase has been indirectly supported by a recent report that minoxidil enhances the expression of vascular endothelial growth factor (VEGF), a growth factor associated with vasodilatation in the dermal papilla which is a main cell making up the hair roots (Br. J. of Dermatol., 1998, 138:407-411). Also, other than the vasodilative effect of the minoxidil in the hair-restoring mechanism, it has been reported that minoxidil promotes activation of dermal papilla cells in the roots of hair incubated in vitro, and growth of hair follicles in a tissue culture of follicles in vitro (Skin Pharmacol., 1996, 9:3-8 and J. Invest. Dermatol., 1989, 92:315-320). These facts indicate that minoxidil may work directly on the roots of hair as a growth factor.

In addition, finasteride, a main component of Propecia which has started to be sold by Merck, is used for treatment of alopecia. It inhibits conversion of the male hormone testosterone into dihydrotestosterone, which is a more potent male hormone than testosterone. On December of 1997, the 1 mg finasteride tablet was approved by the US FDA as a hair-regrowth agent for treatment of male pattern hair loss in men only, and is now commercially available. In clinical studies, it has been demonstrated to have a significant trichogenous effect. However, there has been a report that finasteride may inhibit male sexual function as a side effect (J. Am. Acad Dermatol., 1998, 39:578-589). Since neither finasteride nor minoxidil show superior effect in clinical tests, and there is concern about side effects, many researches are conducted to develop a new and improved hair-regrowth agents.

The cyclosporin family of drugs has immunosuppressive activity. It is also effective to inhibit growth of virus, fungus, protozoan, etc. and has various physiological effects such as nephrotoxicity, hepatotoxicity, hypertension, enlargement of periodontium, trichogenous effect, and so on, as side effects (Advances in Pharmacol., 1996, 35:114-246 and Drug Safety, 1994, 10:310-317). Cyclosporin A, a representative cyclosporin, is a cyclic peptide having the following Chemical Formula, which comprises 11 amino acids, including several N-methyl amino acids and D-alanine at No. 8 residue.

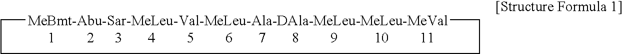

where MeBmt is N-methyl-(4R)-4-[(E)-2-butenyl]-4-methyl-L-threonine, Abu is L-α-aminobutyric acid, Sar is sarcosine, MeLeu is N-methyl-L-leucine, Val is L-valine, Ala is L-alanine, DAla is D-alanine, and MeVal is N-methyl-L-valine.

The amino acid form of cyclosporin A of the above Chemical Formula 1 is L-configuration, unless otherwise specified. The residue numbering of amino acids starts from MeBmt and proceeds clockwise, i.e. 1 for MeBmt and 11 for the last MeVal (N-methyl-L-valine) as shown in the Structure Formula 1. Nomenclature of various derivatives including cyclosporins A to Z, follows methods commonly used (Helv. Chim. Acta, 1987, 70:13-36). For example, if Abu, the residue No. 2 of cyclosporin A is substituted with L-alanine, L-threonine, L-valine or L-norvaline, the derivatives thus prepared are named cyclosporin B, cyclosporin C, cyclosporin D or cyclosporin G, respectively. Further, when the amino acid residues of the cyclosporin derivatives differ from those of cyclosporin A, the derivatives are named by describing the substituent. For example, if cyclosporin A is modified, the derivative is named [γ-hydroxy-N-methyl-L-leucine⁴] cyclosporin A, and if cyclosporin B and cyclosporin C are modified, the derivatives are named [γ-hydroxy-N-methyl-L-leucine⁴] B and [γ-hydroxy-N-methyl-L-leucine⁴] cyclosporin C, respectively. If sarcosine, being the residue No. 3 of cyclosporin A, is substituted with N-methyl-D-Abu³ or N-methyl-D-Nva³, the derivatives thus prepared are named [N-methyl-D-Abu³] cyclosporin A or [N-methyl-D-Nva³] cyclosporin A, respectively. Also, in the case that two or more residues are substituted, the derivatives are named in a similar manner. For example, if both of residues No. 3 and No. 4 are substituted, the derivative is named [N-methyl-D-alanine³][γ-hydroxy-N-methyl-L-leucine⁴] cyclosporin A. If both of residues No. 4 and No. 9 are substituted, the derivative is named [γ-hydroxy-N-methyl-L-leucine⁴][γ-hydroxy-N-methyl-L-leucine⁹] cyclosporin A. Also, if substitution at the residues No. 4 and No. 7 concurs, the derivative is described as [γ-hydroxy-N-methyl-L-leucine⁴][alanine thiomide⁷, [⁷ψ⁸ CS—NH] cyclosporin A.

Meanwhile, a peptolide is produced by conversion of an amide bond to an ester bond in a cyclosporin molecule due to substitution of, for example, the No. 8 residue, D-alanine, with hydroxy acid. The hydroxyl group has a general formula, —O—RCH—CO—, in which R is a C₁₋₄ alkyl group. The most preferred hydroxyl group is hydroxyisovaleric acid (hereinafter abbreviated to Hiv). Representative peptolides include [L-threonine²][L-leucine⁵][D-2-hydroxyisovaleric acid⁸][L-leucine¹⁰] cyclosporin A, [L-Thr²][D-Hiv⁸][Leu¹⁰] cyclosporin A, [L-Thr²][Ile⁵][D-Hiv⁸][Leu¹⁰] cyclosporin A, [L-Thr²][Leu⁴][Leu⁵][D-Hiv⁸][Leu¹⁰] cyclosporin A, and [L-Thr²][Sar³][Leu⁵][D-Hiv⁸][Leu¹⁰] cyclosporin A, capable of being obtained by employing Cylindrotrichum Bonorden NRRL 18230.

Regarding the peptolide in which the residue No. 8, D-alanine is substituted with D-hydroxyisovaleric acid, forming an ester bond in its amino sequence, if the residue No. 4, N-methyl-L-leucine is converted to [γ-hydroxy-N-methyl-L-leucine⁴], the cyclosporin derivative is described as [L-threonine²][L-leucine⁵][γ-hydroxy-N-methyl-L-leucine⁴][D-hydroxyisovaleric acid⁸][L-leucine¹⁰] cyclosporin. Further, as for a derivative of cyclosporin which is substituted with sulfur instead of a carbonyl oxygen at the amino acid residue 7, the name of the derivative may be cyclosporin 7-thioamide or [⁷ψ⁸ CS—NH] cyclosporin, according to different references (Helv. Chim. Acta. 74: 1953-1990, 1991; J. Org. Chem. 58: 673-677, 1993; J. Org. Chem. 59: 7249-7258, 1994). In addition, a common method for abbreviating amino acids is employed, that is, N-methyl-L-leucine is abbreviated by MeLeu, N-methyl-L-isoleucine by MeIle, N-methyl-L-Valine by MeVal, N-methyl-L-alanine by MeAla, N-methyl-L-norvaline by MeNva, L-leucine by Leu, L-isoleucine by Ile, sarcosine by Sar, L-serine by Ser, L-valine, Val, L-alanine by Ala, D-alanine by DAla, L-aminobutyric acid by Abu, L-threonine by Thr, and L-norvaline by Nva. In the invention, the term ‘cyclosporin deriatives’ generally refers to [γ-hydroxy-N-methyl-L-leucine⁴] cylosporin derivatives which are substituted at residue No. 4 of cyclosporin with γ-hydroxy-N-methyl-L-leucine⁴, having an excellent hair restoring effect with non-immunosuppressive activity.

So far, possible development of cyclosporin as a hair-regrowth agent has been studied by many research groups. Particularly, researches involving animal hair regrowth tests (Arch, Dermatol. Res., 1996, 288:408-410), human alopecia areata (J. Am. Acad. Dermatol., 1990, 22:242-250), human male pattern alopecia (J. Am. Acad. Dermatol., 1990, 22:251-253 and Skin Pharmacol., 1994, 7:101-104), and inhibition effect of hair loss by chemotherapy in animal models (Clin. Lab. Invest., 1995, 190:192-196 and Am. J. Pathol., 1997, 150:1433-1441) have been widely conducted. In comparative experiments on mouse's back, it is shown that cyclosporin has a hair regrowth effect about 100 times superior to minoxidil. Based on such findings, there have been attempts to utilize cyclosporin as a treatment for male pattern alopecia, and many applications for patents have been filed.

For example, Japanese Patent Publication Kokai Nos. Sho 60-243008, Sho 62-19512 and Sho 62-19513 disclose use of cyclosporin derivatives as a hair regrowth agent. Also, European Patent Publication No. 0414632 B1 discloses cyclosporin derivatives modified at residue No. 8, and PCT Patent Publication No. WO 93/17039 discloses isocyclosporin provided as a hair regrowth agent. In U.S. Pat. No. 5,807,820 and U.K. Patent No. 2,218,334 A, preparations containing cyclosporin with excellent transdermal absorption are suggested for new application of a hair regrowth agent.

### DISCLOSURE OF THE INVENTION

Therefore, the present invention has been made in view of the above problems associated with side effects of cyclosporin A, and based on the knowledge that the hair restoring effect of cyclosporin is not always in line with its immunosuppressive activity (Iwabuchi et al., Dermatol. Sci., 1995, 9:64-69), and it is an object of the present invention to provide a novel hair growth promoting agent prepared by a variety of molecular modification from cyclosporin, which retains a hair restoring effect, while its immunosuppressive activity is lost.

Approaches similar to the above have been actively made to develop agents for the treatment of acquired immunodeficiency syndrome (AIDS) caused by infection with HIV virus. Those agents are cyclosporin derivatives retaining their inhibition activity against HIV, while having reduced immunosuppressive activity. Esecially, [γ-hydroxy-MeLeu⁴] cyclosporin A, [MeIle⁴] cyclosporin A and [MeVal⁴] cyclosporin A are cyclosporin derivatives with modifications at amino acid residue No. 4, which are disclosed in references as novel anti-HIV agents (U.K. Patent No. 484,281 A2, U.S. Pat. No. 5,767,069, J. Virol., 1995, 69:2451-2461, and J. Antibiotics, 1996, 49:781-787). The inventors examined hair restoring effects and immunosuppressive activities with respect to a variety of cyclosporin derivatives including the derivatives substituted at residue No. 4 with γ-hydroxy methylleucine, methylisoleucine, methylvaline, leucine, or isoleucine, instead of methylleucine, the original residue No. 4, with γ-hydroxy methylleucine being the residue similar in structure to the original MeLeu. Among the above derivatives tested, [γ-hydroxy-N-methyl-L-leucine⁴] cyclosporin A was found to be a novel hair growth promoting agent which uniquely has a hair restoring effect with no immunosuppressive activity. This finding led to searches for derivatives other than cyclosporin A. As one of the results, it was found that [N-methyl-D-alanine³][γ-hydroxy-N-methyl-L-leucine⁴] cyclosporin A also has a hair restoring effect while the immunosuppressive activity is reduced. Accordingly, the invention is directed to [γ-hydroxy-N-methyl-L-leucine⁴] cyclosporin derivatives.

The present invention is directed to a hair growth promoting agent comprising a [γ-hydroxy-N-methyl-L-leucine⁴] cyclosporin derivative represented by Chemical Formula 1 below, having an excellent hair restoring effect with non-immunosuppressive activity, as an active ingredient.

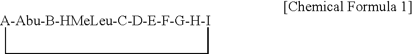

wherein:

A represents N-methyl-(4R)-4-[(E)-2-butenyl]-4-methyl-L-threonine (MeBmt), (2S,3R,4R,6E)-3-sulfhydryl-4-methyl-2-(methylamino)-6-octenoic acid or (2S,4R,6E)-3-oxo-4-methyl-2-(methylamino)-6-octenoic acid;

Abu represents L-γ-aminobutyric acid (Abu);

B represents a D-amino acid represented by the general formula 1,

CH₃NH—CH(R)—COOH [General formula 1]

in which,

R is one selected from the group consisting of hydrogen, C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino, and X—R! represented by the general formula 2 below,

—X—R′ [General formula 2]

in which,

X is oxygen or sulfur, and

R′ is one selected from the group consisting of hydrogen, and C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino;

C represents L-valine or L-norvaline;

D represents N-methyl-L-leucine, γ-hydroxy-N-methyl-L-leucine or L-leucine;

E represents L-alanine or L-alanine thioamide ([⁷ψ⁸ CS—NH], NH—CHCH₃—CS—);

F represents D-2-hydroxyisovaleric acid or a D-amino acid represented by the general formula 3,

—NH—CH(CH₂R)—COOH [General formula 3]

in which,

R is hydrogen or X—R′ represented by the general formula 4,

—X—R′ [General formula 4]

in which,

X is oxygen or sulfur, and

R′ is one selected from the group consisting of hydrogen, and C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino;

G represents N-methyl-L-leucine, γ-hydroxy-N-methyl-L-leucine or L-leucine;

H represents N-methyl-L-leucine, γ-hydroxy-N-methyl-L-leucine or L-leucine; and

I represents N-methyl-L-valine or L-valine.

The preferred derivative of the above Chemical Formula 1, having an excellent hair restoring effect with non-immunosuppressive activity, is a [γ-hydroxy-N-methyl-L-leucine⁴] cyclosporin derivative represented by Chemical Formula 2.

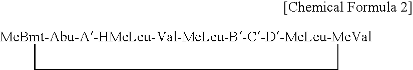

wherein:

MeBmt represents N-methyl-(4R)-4-[(E)-2-butenyl]-4-methyl-L-threonine;

Abu represents L-α aminobutyric acid (Abu);

A′ represents N-methyl-D-alanine, D-2-(methylamino)pent-4-enoyl, N-methyl-D-aminobutyric acid, N-methyl-D-norvaline, D-2-(methylamino)hexa-4-ynoyl, D-2-(methylamino)pent-4-ynoyl, D-2-methylthio-sarcosine, N-methyl-O-propenyl-D-serine or N-methyl-D-serine;

HMeLeu represents γ-hydroxy-N-methyl-L-leucine;

Val represents L-valine;

MeLeu represents N-methyl-L-leucine;

B′ represents L-alanine or L-alanine thioamide ([⁷ψ⁸ CS—NH], NH—CHCH₃—CS—);

C′ represents D-2-hydroxyisovaleric acid, or a D-amino acid represented by the general formula 3,

—NH—CH(CH₂R)—COOH [General formula 3]

in which,

R is hydrogen or X—R′ represented by the general formula 4,

—X—R′ [General formula 4]

in which,

X is oxygen or sulfur, and

R′ is one selected from the group consisting of hydrogen, and C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino;

D′ represents N-methyl-L-leucine, γ-hydroxy-N-methyl-L-leucine or L-leucine; and

MeVal represents N-methyl-L-valine.

The more preferred derivative of the above Chemical Formula 1, having an excellent hair restoring effect with non-immunosuppressive activity, is a [γ-hydroxy-N-methyl-L-leucine⁴] cyclosporin derivative represented by Chemical Formula 3.

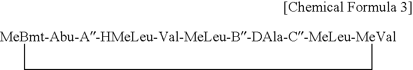

wherein:

MeBmt represents N-methyl-(4R)-4-[(E)-2-butenyl]-4-methyl-L-threonine;

Abu represents L-α aminobutyric acid (Abu);

A″ represents N-methyl-D-alanine, D-2-(methylamino)pent-4-enoyl, N-methyl-D-aminobutyric acid, N-methyl-D-norvaline, D-2-(methylamino)hexa-4-ynoyl, D-2-(methylamino)pent-4-ynoyl, D-2-methylthio-sarcosine, N-methyl-O-propenyl-D-serine or N-methyl-D-serine;

HMeLeu represents γ-hydroxy-N-methyl-L-leucine;

Val represents L-valine;

MeLeu represents N-methyl-L-leucine;

B″ represents L-alanine or L-alanine thioamide ([⁷ψ⁸ CS—NH], NH—CHCH₃—CS—);

DAla represents D-alanine;

C″ represents N-methyl-L-leucine, γ-hydroxy-N-methyl-L-leucine or L-leucine; and

MeVal represents N-methyl-L-valine.

In accordance with another aspect of the present invention, there is provided a hair growth promoting agent comprising [N-methyl-D-alanine³][γ-hydroxy-N-methyl-L-leucine⁴] cyclosporin A as an active ingredient.

In accordance with another aspect of the present invention, there is provided a hair growth promoting agent comprising [D-2-(methylamino)pent-4-enoyl³][γ-hydroxy-N-methyl-L-leucine⁴] cyclosporin A as an active ingredient.

In accordance with another aspect of the present invention, there is provided a hair growth promoting agent comprising [N-methyl-D-Abu³][, γ-hydroxy-N-methyl-L-leucine⁴] cyclosporin A as an active ingredient.

In accordance with another aspect of the present invention, there is provided a hair growth promoting agent comprising [N-methyl-D-Norvaline-3][γ-hydroxy-N-methyl-L-leucine⁴] cyclosporin A as an active ingredient.

In accordance with another aspect of the present invention, there is provided a hair growth promoting agent comprising [D-2-(methylamino)hexa-4-ynoyl³][γ-hydroxy-N-methyl-L-leucine⁴] cyclosporin A as an active ingredient.

In accordance with another aspect of the present invention, there is provided a hair growth promoting agent comprising [D-2-(methylamino)pent-4-ynoyl³][γ-hydroxy-N-methyl-L-leucine⁴] cyclosporin A as an active ingredient.

In accordance with another aspect of the present invention, there is provided a hair growth promoting agent comprising [D-2-methylamino-Sar³][γ-hydroxy-N-methyl-L-leucine⁴] cyclosporin A as an active ingredient.

In accordance with another aspect of the present invention, there is provided a hair growth promoting agent comprising [N-methyl-O-propenyl-D-serine³][γ-hydroxy-N-methyl-L-leucine⁴] cyclosporin A as an active ingredient.

In accordance with another aspect of the present invention, there is provided a hair growth promoting agent comprising [N-methyl-D-serine³][γ-hydroxy-N-methyl-L-leucine⁴] cyclosporin A as an active ingredient.

In accordance with another aspect of the present invention, there is provided a hair growth promoting agent comprising [γ-hydroxy-N-methyl-L-leucine⁴][alanine thioamide⁷, ⁷ψ⁸ CS—NH] cyclosporin A as an active ingredient.

In accordance with another aspect of the present invention, there is provided a hair growth promoting agent comprising [γ-hydroxy-N-methyl-L-leucine⁴][γ-hydroxy-N-methyl-L-leucine⁹] cyclosporin A as an active ingredient.

In accordance with another aspect of the present invention, there is provided a hair growth promoting agent comprising [γ-hydroxy-N-methyl-L-leucine⁴][D-serine⁸] cyclosporin A as an active ingredient.

In accordance with another aspect of the present invention, there is provided a hair growth promoting agent comprising [L-threonine²][γ-hydroxy-N-methyl-L-leucine⁴][L-leucine⁵][D-2-hydroxyisovaleric acid⁸][L-leucine¹⁰] cyclosporin A as an active ingredient.

In accordance with yet another aspect of the present invention, there is provided a hair growth promoting agent whose composition comprising a [γ-hydroxy-N-methyl-L-leucine⁴] cyclosporin derivative may be formulated in the form of liquid formulations, sprays, gels, pastes, emulsions, creams, conditioners or shampoos.

### BRIEF DESCRIPTION OF THE DRAWINGS

The above and other objects, features and other advantages of the present invention will be more clearly understood from the following detailed description taken in conjunction with the accompanying drawing, in which:

FIG. 1 is a HPLC chromatogram of [N-methyl-D-alanine³][γ-hydroxy-N-methyl-L-leucine⁴] cyclosporin A (having a retention time of 18 to 20 min);

FIG. 2 is a ¹H-NMR spectrum of [N-methyl-D-alanine³][γ-hydroxy-N-methyl-L-leucine⁴] cyclosporin A;

FIG. 3 is a ¹³C-NMR spectrum of [N-methyl-D-alanine³][γ-hydroxy-N-methyl-L-leucine⁴] cyclosporin A;

FIG. 4 is a HPLC chromatogram of [γ-hydroxy-N-methyl-L-leucine⁴][alanine thioamide⁷, ⁷ψ⁸ CS—NH] cyclosporin A (having a retention time of around 16.2 min);

FIG. 5 is a ¹H-NMR spectrum of [γ-hydroxy-N-methyl-L-leucine⁴][alanine thioamide⁷, ⁷ψ⁸ CS—NH] cyclosporin A;

FIG. 6 is a ¹³C-NMR spectrum of [γ-hydroxy-N-methyl-L-leucine⁴][alanine thioamide⁷, ⁷ψ⁸ CS—NH] cyclosporin A;

FIG. 7 is a ¹H-NMR spectrum of [γ-hydroxy-N-methyl-L-leucine⁴][γ-hydroxy-N-methyl-L-leucine⁹] cyclosporin A;

FIG. 8 is a ¹³C-NMR spectrum of [γ-hydroxy-N-methyl-L-leucine⁴][γ-hydroxy-N-methyl-L-leucine⁹] cyclosporin A;

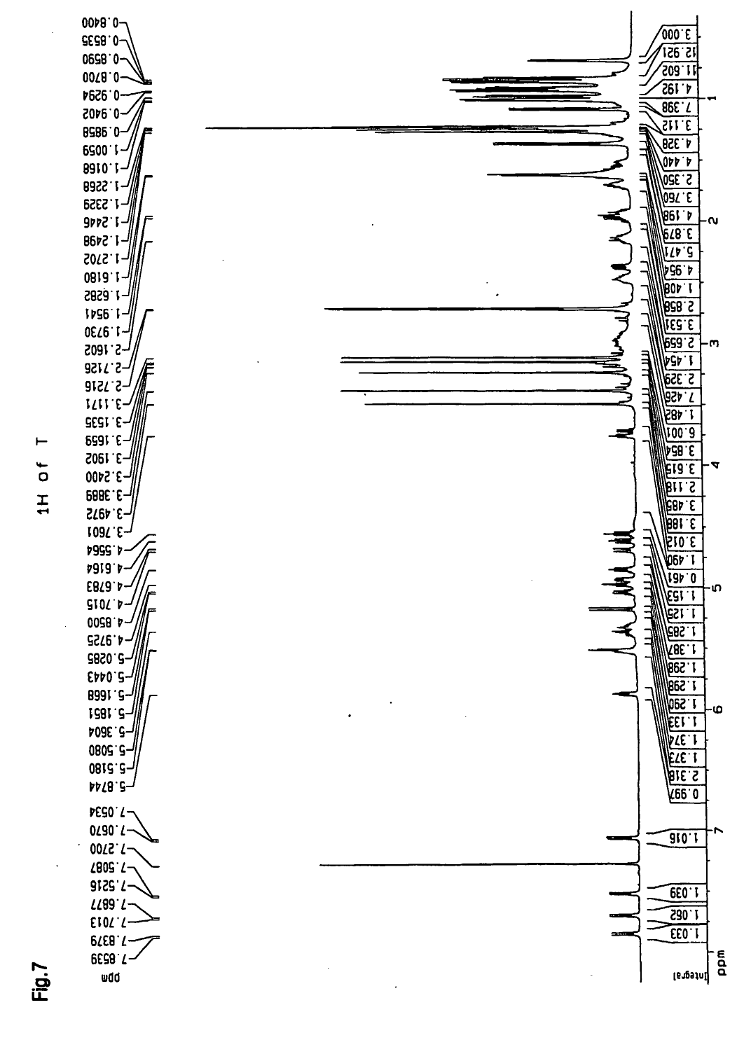

FIG. 7 is a ¹H-NMR spectrum of [D-2-(methylamino)pent-4-ynoyl³] cyclosporin A;

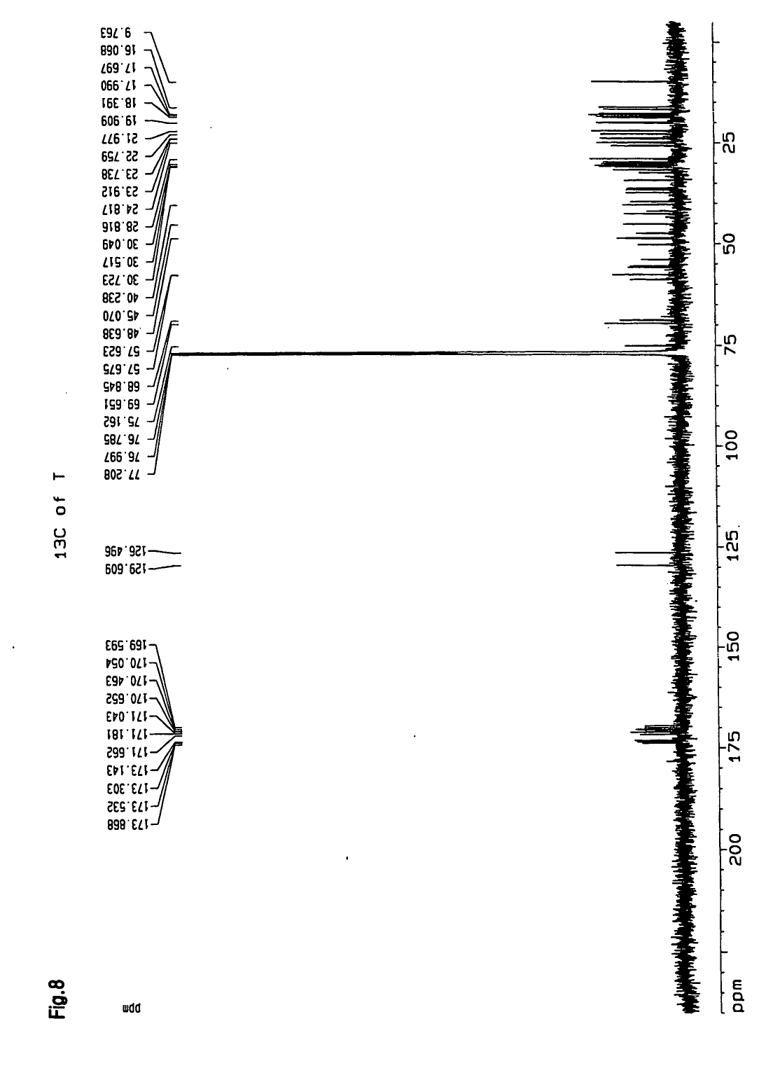

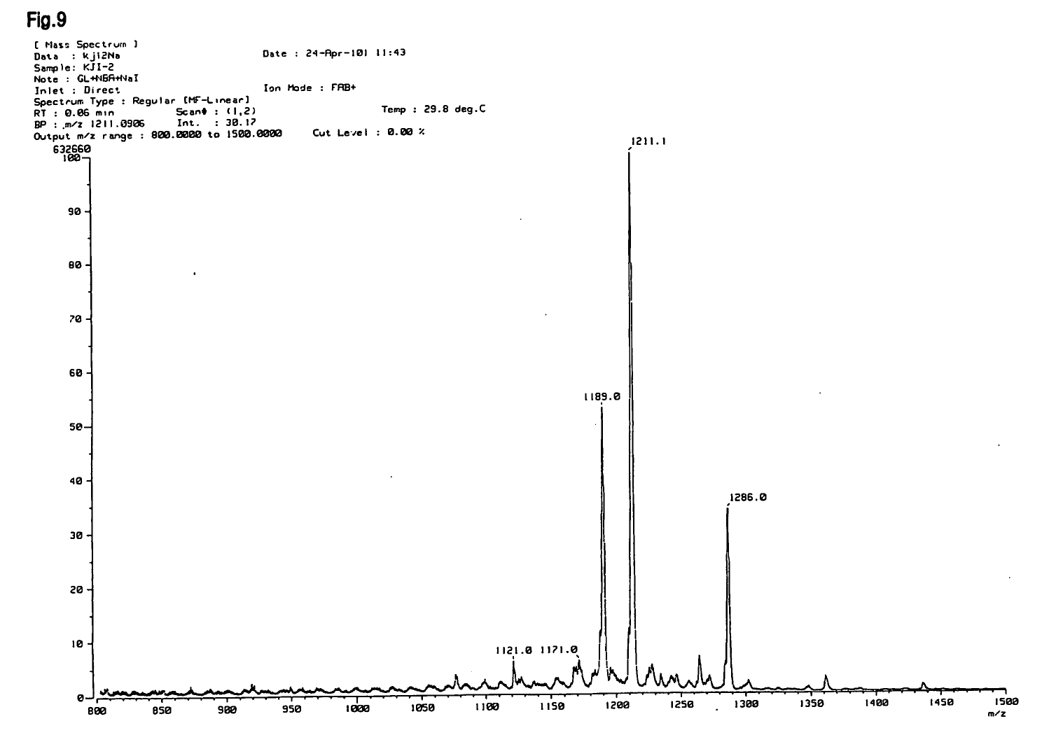

FIG. 8 is a ¹³C-NMR spectrum of [D-2-(methylamino)pent-4-ynoyl³] cyclosporin A; and

FIG. 9 is a FAB-MS spectrum of [L-threonine²][γ-hydroxy-N-methyl-L-leucine⁴][L-leucine⁵][D-2-hydroxyisovaleric acid⁸][L-leucine¹⁰] cyclosporin A with a peak at m/z 1286.0 representing [M (peptolide derivative)+Na].

### BEST MODE FOR CARRYING OUT THE INVENTION

Hereinafter, the present invention will be described in detail, in conjunction with various examples. These examples are provided only for illustrative purposes, and the present invention is not to be construed as being limited to those examples.

With the aim of developing a novel hair growth promoting agent having a hair restoring effect without immunosuppressive activity, the present inventors prepared a variety of cyclosporin derivatives via chemical synthesis and modification, and hair growth promoting effects thereof were examined. As a result, most of cyclosporin derivatives showed a considerable decrease in their hair restoring effects, compared to unmodified cyclosporin. However, [γ-hydroxy-N-methyl-L-leucine⁴] cyclosporin derivatives, which are prepared by modifying cyclosporin using microorganisms, retain their hair restoring effects, while having no immunosuppressive activity.

### EXAMPLE 1

Preparation of [N-methyl-D-alanine³][γ-hydroxy-N-methyl-L-leucine⁴] cyclosporin A

Step 1-1: General Method for Alkylation of Cyclosporin A

Tetrahydrofuran (THF) was added with diisopropyl amine ((i-Pr)₂NH) and added with a solution of n-butyl lithium (BuLi) in hexane under a nitrogen atmosphere at −78° C., followed by stirring for 30 min. To the solution of LDA (lithium diisopropylamide) thus prepared, cyclosporin A in THF was added, stirred for 1 hr, and electrophile was added.

Step 1-2: Synthesis of [D-MeAla³] Cyclosporin A

According to the general method, alkylation was performed employing THF (120 ml), (i-Pr)₂NH (1.74 ml), BuLi (4.6 ml), cyclosporin A (2.0 g) in 30 ml THF, and methyliodide (MeI) (0.51 ml). The solution was warmed to room temperature while stirring for 1 hr, and added with 10 ml water, followed by concentration. The residue was added with ether (Et₂O), washed with water and a saturated aqueous sodium chloride solution in sequence, and dried over anhydrous MgSO₄. After concentrating, the residue was subjected to silica gel column chromatography (100 g silica gel, dichloromethane:methylalcohol=50:1˜96: 4), followed by HPLC to give the title compound (0.26 g).

Step 1-3: Preparation of [N-methyl-D-alanine³][γ-hydroxy-N-methyl-L-leucine⁴] cyclosporin A

Herein is described a method of preparing [γ-hydroxy-N-methyl-L-leucine⁴] cyclosporin A, which retains a hair restoring effect after modification by microorganisms. As a bacterial strain for preparing the cyclosporin derivative, Sebekia benihana KCTC 9173 was used. The culture medium contained 0.7% glucose, 0.45% yeast extract, 0.5% malt extract, 1.0% soluble starch, and 0.005% CaCO₃, and the incubation temperature was 27° C. (J. Antibiotics, 1996, 49:781-787). After being first pre-cultured for 4 days in an Erlenmeyer flask, the bacterial strain was cultured in the above medium using a 4 L fermentor.

Then, [D-MeAla³] cyclosporin A in methanol was added at 100 mg/L to the culture medium 24 hrs after starting the main culture. The bacteria were further cultured for 72 hrs.

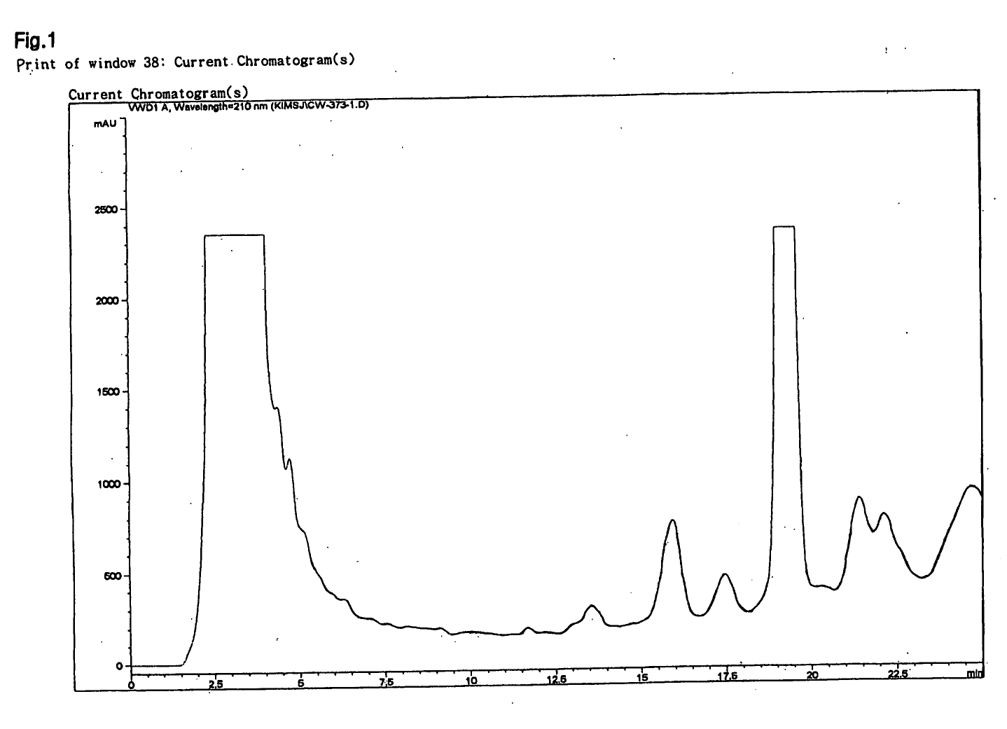

After 72 hrs, the total culture solution was extracted with an equal volume of ethyl acetate, thereby collecting the sample. The organic solvent layer was concentrated. The concentrated sample was subjected to liquid chromatograhy, isolating and collecting cyclosporin derivatives. The results of liquid chromatography are represented in FIG. 1, which shows [D-MeAla³] cyclosporin A and [N-methyl-D-alanine³][γ-hydroxy-N-methyl-L-leucine⁴] cyclosporin A derivatives (having a retention time of 18 to 20 min). The isolation condition was as follows. A C-18 column was used. For the elution, a 100% solvent A was flowed for 2 min. The concentration of the solvent was lowered to 60%, and the 60% solvent was flowed over 4 min, and then the concentration was slowly lowered to 39%, over 60 min. The concentration of the solvent was then returned to 100%, and flowed for a further 5 min. The solvent A was a 25% aqueous methanol solution. As the diluent solvent B, 100% acetonitrile was used.

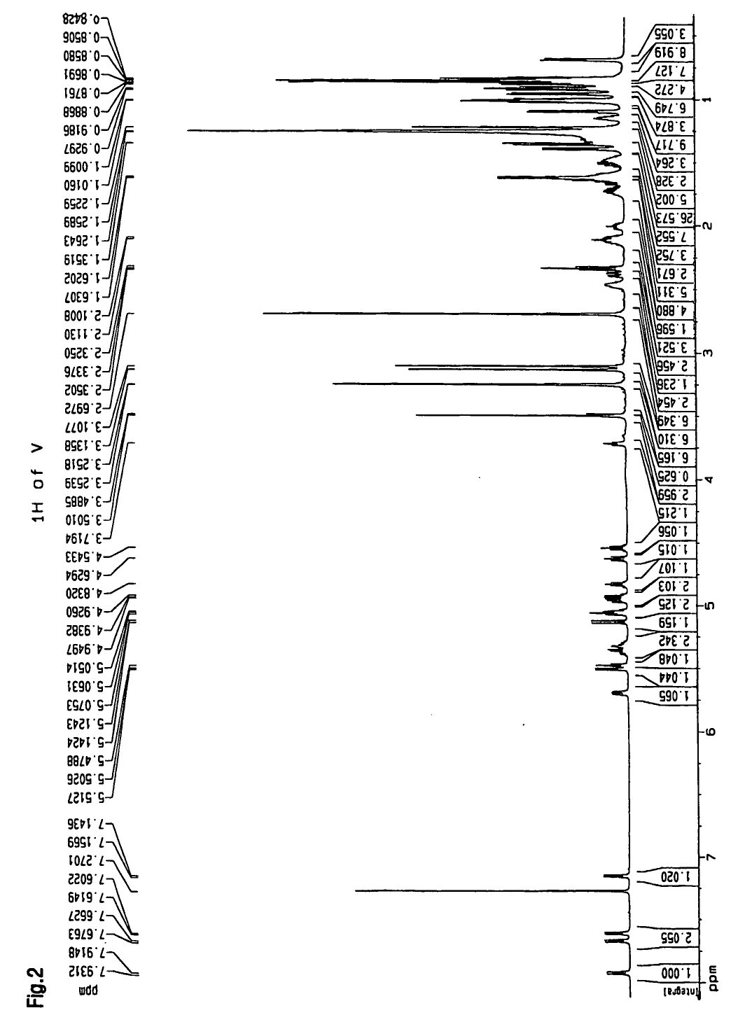

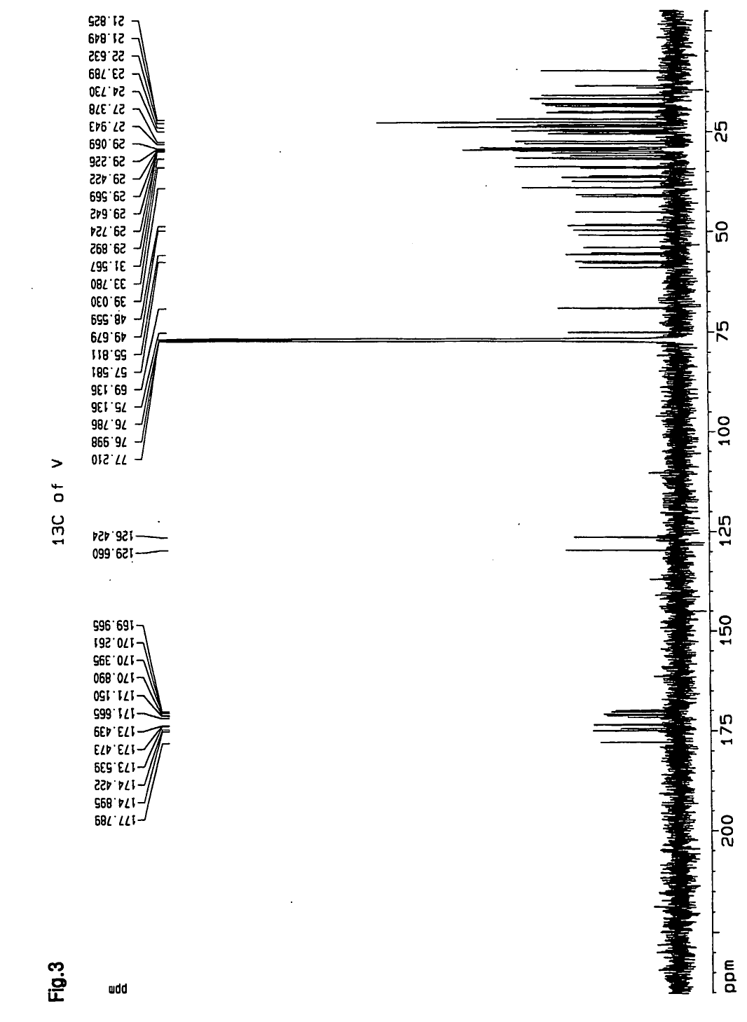

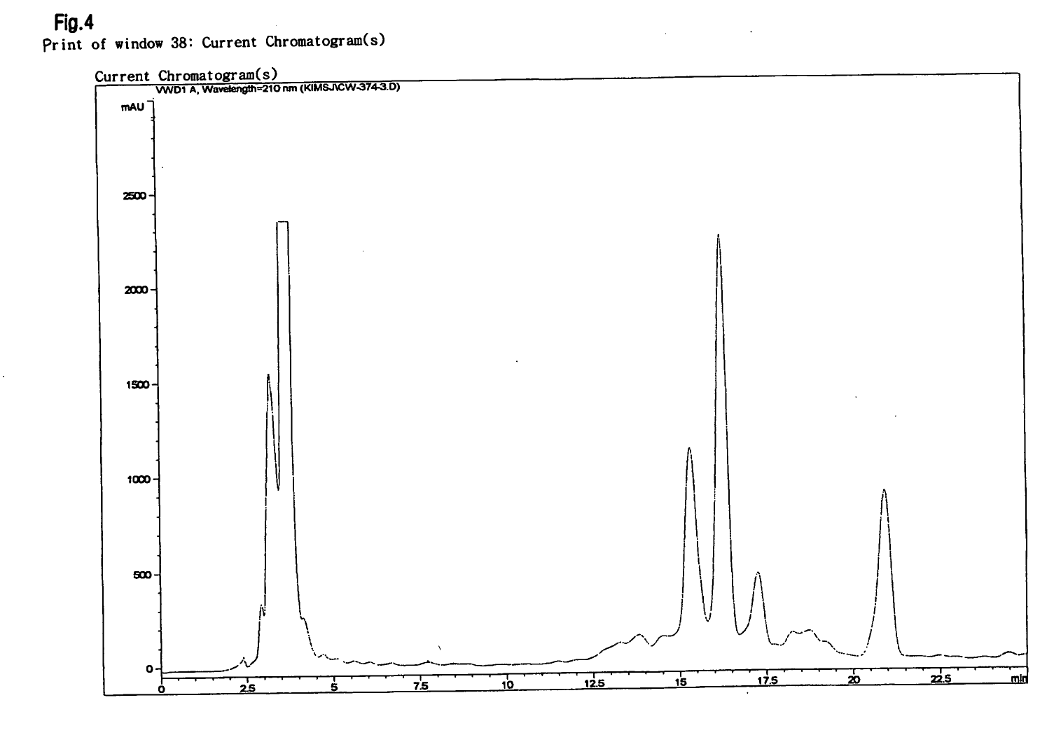

Molecular weight of the compound was determined by FAB MS (ZMS AX 505H) analysis. To confirm the molecular structure, Nuclear Magnetic Resonance (NMR) measurements were performed on 600 MHz (Bruker) for ¹H-NMR and on 150 MHz (Bruker) for ¹³C-NMR, and the spectra are shown in FIGS. 2 and 3, respectively.

### EXAMPLE 2

Preparation of [γ-hydroxy-N-methyl-L-leucine⁴][alanine thiomide⁷, ⁷ψ⁸ CS—NH] cyclosporin A

Step 2-1: Synthesis of Acetylcyclosporin A

2.4 g (2.0 mmol) of cyclosporin A and 0.24 g (2.0 mmol) of 4-(dimethylamino)pyridine were added to 20 ml of pyridine and 20 ml of acetic anhydride, stirred for 18 hours at room temperature, and distilled under reduced pressure. To the residue was added 100 ml of methylene chloride. The residue was then washed with water and dried over anhydrous magnesium sulfate (MgSO₄). The crude product was purified by chromatography on a silica gel column to give 2.3 g of the title compound.

Step 2-2: Synthesis of Acetylcyclosporin A 7-thioamide

1.8 g (1.45 mmol) of acetylcyclosporin was dissolved in 50 ml of xylene. The resulting solution was heated to 130° C., and 0.35 g (0.87 mmol) of Lawesson reagent was added in small amounts thereto. The reaction solution was stirred for 30 minutes at 130° C., cooled to room temperature, and distilled under reduced pressure to remove the solvent. To the residue was added 100 ml of methylene chloride. The residue was then washed with water and dried over anhydrous magnesium sulfate (MgSO₄) to form a crude product. The crude product was purified by chromatography on a silica gel column to give 0.19 g of an acetoxy compound, i.e. the title compound.

Step 2-3: Synthesis of Cyclosporin A 7-thioamide

0.2 g (0.16 mmol) of acetylcyclosporin A7-thioamide, the acetoxy compound, was dissolved in 50 ml of methyl alcohol (MeOH). The solution was added with sodium methoxide (NaOMe in MeOH) and stirred for 4 hrs at room temperature. After neutralizing with acetic acid, the resulting solution was distilled under reduced pressure to remove the solvent. To the residue was added 100 ml of methylene chloride. The residue was then washed with water and dried over anhydrous magnesium sulfate (MgSO₄) to form a crude product. The crude product was purified by chromatography on a silica gel column to give 0.17 g of the title compound.

Step 2-4: Preparation of [γ-hydroxy-N-methyl-L-leucine⁴][alanine thiomide⁷, ⁷ψ⁸ CS—NH] cyclosporin A

The bacterial strain used for preparing the cyclosporin derivative was Sebekia benihana KCTC 9173. The culture medium contained 0.7% glucose, 0.45% yeast extract, 0.5% malt extract, 1.0% soluble starch, and 0.005% CaCO₃, and the incubation temperature was 27° C. (J. Antibiotics, 1996, 49:781-787). After being first pre-cultured for 4 days in an Erlenmeyer flask, the bacterial strain was cultured in the above medium using a 4 L fermentor.

Then, cyclosporin A 7-thioamide in methanol was added at 100 mg/L to the culture medium 24 hrs after starting the main culture. The bacteria were further cultured for 72 hrs.

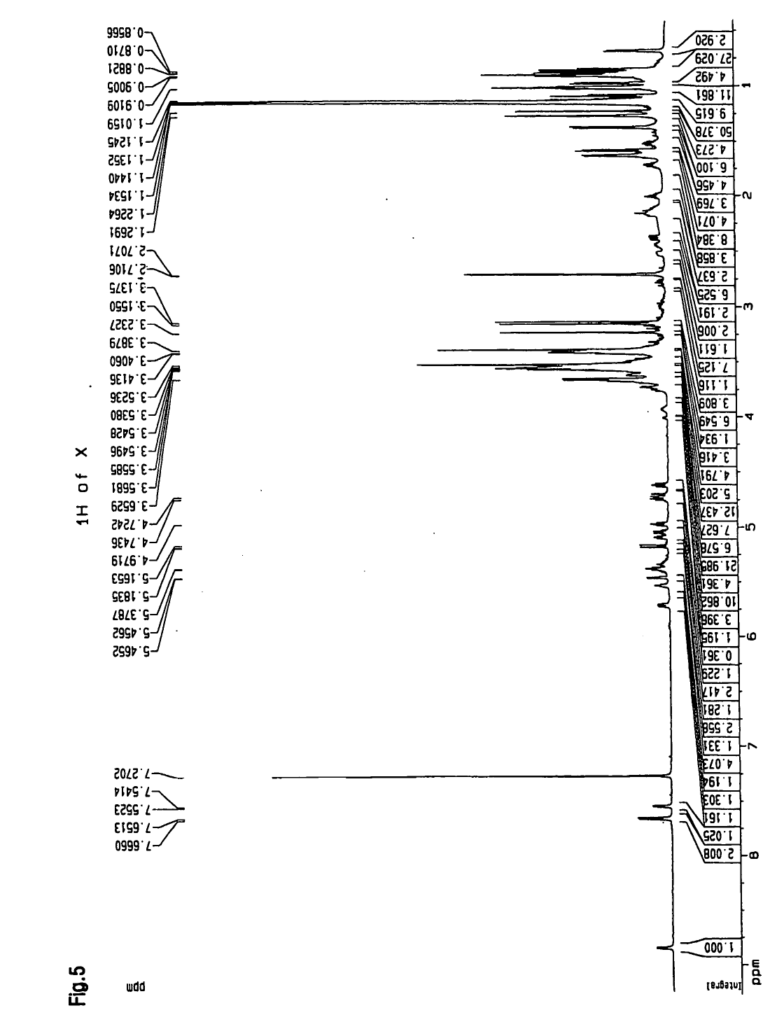

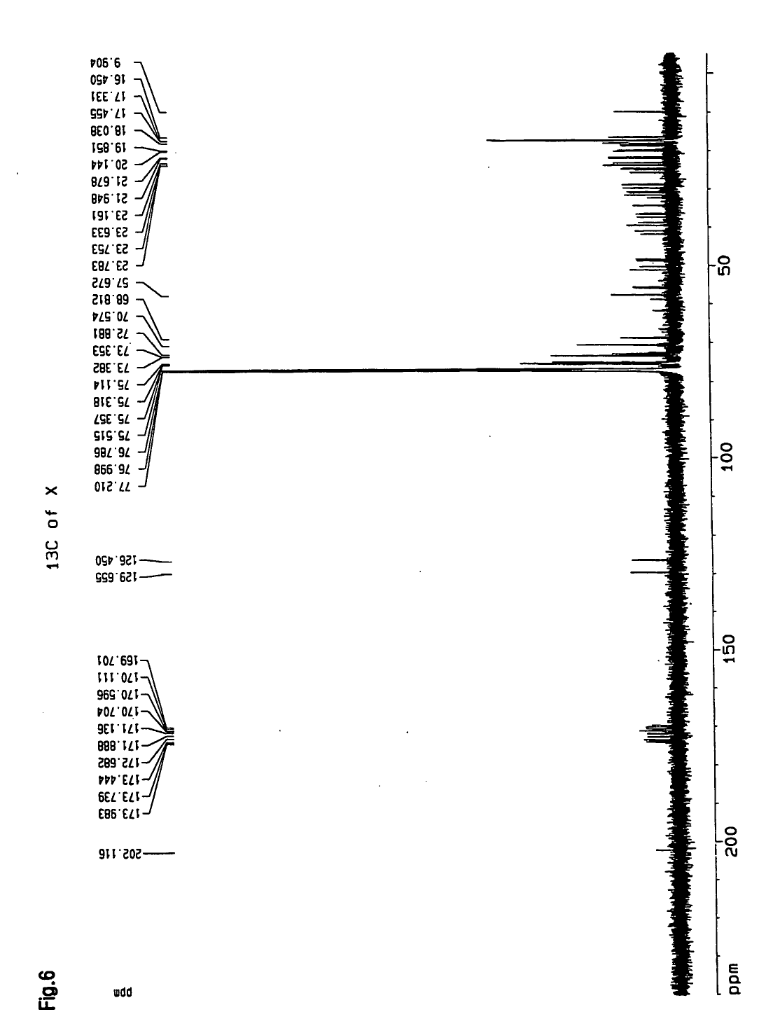

After 72 hrs, the total culture solution was extracted with an equal volume of ethyl acetate, thereby collecting the sample. The organic solvent layer was concentrated. The concentrated sample was subjected to liquid chromatograhy, isolating and collecting cyclosporin derivatives. The results of liquid chromatography are represented in FIG. 4, which shows cyclosporin A 7-thioamide and [γ-hydroxy-N-methyl-L-leucine⁴][alanine thiomide⁷, ⁷ψ⁸ CS—NH] cyclosporin A (having a retention time of around 16.2 min). The isolation condition was as follows. A C-18 column was used. For the elution, a 100% solvent A was flowed for 2 min. The concentration of the solvent was lowered to 60%, and the 60% solvent was flowed over 4 min, and then the concentration was slowly lowered to 39%, over 60 min. The concentration of the solvent was then returned to 100%, and flowed for a further 5 min. The solvent A was a 25% aqueous methanol solution. As the diluent solvent B, 100% acetonitrile was used. Molecular weight of the compound was determined by FAB MS (ZMS AX 505H) analysis. To confirm the molecular structure, Nuclear Magnetic Resonance (NMR) measurements were performed on 600 MHz (Bruker) for ¹H-NMR and on 150 MHz (Bruker) for ¹³C-NMR, and the spectra are shown in FIGS. 5 and 6, respectively.

### EXAMPLE 3

Preparation of [γ-hydroxy-N-methyl-L-leucine⁴][γ-hydroxy-N-methyl-L-leucine⁹] cyclosporin A

As a bacterial strain for preparing the cyclosporin derivative, Pseudonocardia autotrophica KCTC 9441 was used. The culture medium contained 0.7% glucose, 0.45% yeast extract, 0.5% malt extract, 1.0% soluble starch, and 0.005% CaCO₃, and the incubation temperature was 27° C. (J. Antibiotics, 1996, 49: 781-787). After being first pre-cultured for 4 days in an Erlenmeyer flask, the bacterial strain was cultured in the above medium using a 4 L fermentor. Then, cyclosporin A in methanol was added at 100 mg/L to the culture medium 24 hrs after starting the main culture. The bacteria were further cultured for 72 hrs.

After 72 hrs, the total culture solution was extracted with an equal volume of ethyl acetate, thereby collecting the sample. The organic solvent layer was concentrated. The concentrated sample was subjected to liquid chromatograhy, isolating and collecting the cyclosporin derivative. The isolation condition was as follows. A C-18 column was used. For the elution, a 100% solvent A was flowed for 2 min. The concentration of the solvent was lowered to 60%, and the 60% solvent was flowed over 4 min, and then the concentration was slowly lowered to 39%, over 60 min. The concentration of the solvent was then returned to 100%, and flowed for a further 5 min. The solvent A was a 25% aqueous methanol solution. As the diluent solvent B, 100% acetonitrile was used. Molecular weight of the compound was determined by FAB MS (ZMS AX 505H) analysis. To confirm the molecular structure, Nuclear Magnetic Resonance (NMR) measurements were performed on 600 MHz (Bruker) for ¹H-NMR and on 150 MHz (Bruker) for ³C-NMR, and the spectra are shown in FIGS. 7 and 8, respectively.

Alternatively, [γ-hydroxy-N-methyl-L-leucine⁴][γ-hydroxy-N-methyl-L-leucine⁹] cyclosporin A can be prepared using microsomal enzyme obtained from rabbit's liver. First, a liver was removed from a New Zealand White rabbit and immersed in 0.1 M potassium phosphate buffer for 5 min. The liver tissue was minced and homogenized, followed by centrifuging (9000×g, 4° C., 20 min). The supernatant was taken and centrifuged again (10,500×g, 1 hr). After discarding the supernatant, the pellet was dissolved in 0.1 M phosphate buffered saline, to serve as a source of the enzyme (Transplant Proc., 17: 633-636, 1988). The microsomal enzyme (50 mg) thus prepared, cyclosporin (1 mg) and NADPH (5 mM) were mixed with water, making a desired amount of solution. The solution was reacted at 37° C. in water bath for 1 hr. The reaction mixture was extracted with an equal volume of ethyl acetate, followed by analysis.

### EXAMPLE 4

Preparation of [L-threonine²][γ-hydroxy-N-methyl-L-leucine⁴][L-leucine⁵][D-2-hydroxyisovaleric acid⁸][L-leucine¹⁰] cyclosporin A

Step 4-1: Preparation of [L-threonine²][L-leucine⁵][D-2-hydroxyisovaleric acid⁸][L-leucine¹⁰] cyclosporin A peptolide

A fungal strain, Cylindrotrichum Bonorden NRRL 18230 was used (Biomed. Biochim. Acta., 10/11, S260-S263, 1991). The culture medium contained 0.7% glucose, 0.45% yeast extract, 0.5% malt extract, 1.0% soluble starch, and 0.005% CaCO₃, and the incubation temperature was 27° C. After being first pre-cultured for 4 days in an Erlenmeyer flask, the strain was cultured in the above medium using a 4 L fermentor.

After being cultured, the total culture solution was extracted with an equal volume of ethyl acetate, thereby collecting the sample. The organic solvent layer was concentrated. The concentrated sample was subjected to liquid chromatograhy, isolating and collecting the cyclosporin derivative. The isolation condition was as follows. A C-18 column was used. For the elution, a 100% solvent A was flowed for 2 min. The concentration of the solvent was lowered to 60%, and the 60% solvent was flowed over 4 min, and then the concentration was slowly lowered to 39%, over 60 min. The concentration of the solvent was then returned to 100%, and flowed for a further 5 min. The solvent A was a 25% aqueous methanol solution. As the diluent solvent B, 100% acetonitrile was used.

Step 4-2: Preparation of [L-threonine²][γ-hydroxy-N-methyl-L-leucine⁴][L-leucine⁵][D-2-hydroxyisovaleric acid⁸][L-leucine¹⁰] cyclosporin A

For preparing the cyclosporin derivative, Sebekia benihana KCTC 9173 strain was used. The culture medium contains 0.7% glucose, 0.45% yeast extract, 0.5% malt extract, 1.0% soluble starch, and 0.005% CaCO₃, and the incubation temperature was 27° C. (J. Antibiotics, 1996, 49:781-787). Upon culturing using a fermentor, a pre-culture was first performed for 4 days in an Erlenmeyer flask. After pre-culture, the bacterial strain was cultured in the above medium using a 4 L fermentor.

Then, [L-threonine²][L-leucine⁵][D-2-hydroxyisovaleric acid⁸][L-leucine¹⁰] cyclosporin A peptolide in methanol was added at 100 mg/L to the culture medium 24 hrs after starting the main culture. The bacteria were further cultured for 72 hrs.

After 72 hrs, the total culture solution was extracted with an equal volume of ethyl acetate, thereby collecting the sample. The organic solvent layer was concentrated. The concentrated sample was subjected to liquid chromatograhy, isolating and collecting the cyclosporin derivative. The isolation condition was as follows. A C-18 column was used. For the elution, a 100% solvent A was flowed for 2 min. The concentration of the solvent was lowered to 60%, and the 60% solvent was flowed over 4 min, and then the concentration was slowly lowered to 39%, over 60 min. The concentration of the solvent was then returned to 100%, and flowed for a further 5 min. The solvent A was a 25% aqueous methanol solution. As the diluent solvent B, 100% acetonitrile was used. The molecular weight of [L-threonine²][γ-hydroxy-N-methyl-L-leucine⁴][L-leucine⁵][D-2-hydroxyisovaleric acid⁸][L-leucine¹⁰] cyclosporin A was determined by FAB MS (ZMS AX 505H) analysis. [M (peptolide derivative)+Na] peak was observed at m/z 1286.0 (FIG. 9). To confirm the molecular structure, Nuclear Magnetic Resonance (NMR) measurements were performed on 600 MHz (Bruker) for ¹H-NMR and on 150 MHz (Bruker) for ¹³C-NMR.

Formulations

Formulation 1-1:

Preparation of hair tonic containing [N-methyl-D-alanine³][γ-hydroxy-N-methyl-L-leucine⁴] cyclosporin A

Individual ingredients were mixed and stirred, and the mixtures were completely dissolved to prepare three hair growth promoting tonics, with compositions as shown in Table 1 below. It was found that the composition 1 of Table 1 has a hair growth promoting effect at a level similar to a conventional hair tonic containing 0.1% cyclosporin A, as evaluated in an animal experiment according to Test Example 1 described later.

TABLE 1
Formulation of hair tonic

| Ingredients | Comp. 1 | Comp. 2 | Comp. 3 |
|---|---|---|---|
| ethanol | 40.0 | 40.0 | 40.0 |
| [N-methyl-D-alanine³] | 0.1 | 1.0 | 8.0 |
| [γ-hydroxy-N-methyl- | | | |
| L-leucine⁴]cyclosporin A | | | |
| tocopherol acetate | 0.1 | 0.1 | 0.1 |
| salicylic acid | 0.3 | 0.3 | 0.3 |
| L-menthol | 0.3 | 0.3 | 0.3 |
| Tween 20 | 0.5 | 0.5 | 0.5 |
| perfume | typical | typical | typical |
| colorant | typical | typical | typical |
| water | balance | balance | balance |

(unit: weight%)

Formulation 1-2:

Preparation of hair tonic containing [γ-hydroxy-N-methyl-L-leucine⁴][alanine thiomide⁷, ⁷ψ⁸ CS—NH] cyclosporin A

Individual ingredients were mixed and stirred, and the mixtures were completely dissolved to prepare three hair growth promoting tonics, with compositions as shown in Table 2 below. It was found that the composition 1 of Table 2 has a hair growth promoting effect at a level similar to a conventional hair tonic containing 0.1% cyclosporin A, as evaluated in an animal experiment according to Test Example 1 described later.

TABLE 2
Formulation of hair tonic

| Ingredients | Comp. 1 | Comp. 2 | Comp. 3 |
|---|---|---|---|
| ethanol | 40.0 | 40.0 | 40.0 |
| [γ-hydroxy-N-methyl-L- | 0.1 | 1.0 | 8.0 |
| leucine⁴][alanine | | | |
| thiomide⁷,⁷Ψ⁸CS—NH] | | | |
| cyclosporin A | | | |
| tocopherol acetate | 0.1 | 0.1 | 0.1 |
| salicylic acid | 0.3 | 0.3 | 0.3 |
| L-menthol | 0.3 | 0.3 | 0.3 |
| Tween 20 | 0.5 | 0.5 | 0.5 |
| perfume | typical | typical | typical |
| colorant | typical | typical | typical |
| water | balance | balance | balance |

(unit: weight%)

Formulation 1-3:

Preparation of hair tonic containing [γ-hydroxy-N-methyl-L-leucine⁴][γ-hydroxy-N-methyl-L-leucine⁹] cyclosporin A

Individual ingredients were mixed and stirred, and the mixtures were completely dissolved to prepare three hair growth promoting tonics, with compositions as shown in Table 3 below. It was found that the composition 1 of Table 3 has a hair growth promoting effect at a level similar to a conventional hair tonic containing 0.1% cyclosporin A, as evaluated in an animal experiment according to Test Example 1 described later.

TABLE 3
Formulation of hair tonic

| Ingredients | Comp. 1 | Comp. 2 | Comp. 3 |
|---|---|---|---|
| ethanol | 40.0 | 40.0 | 40.0 |
| [γ-hydroxy-N-methyl- | 0.1 | 1.0 | 8.0 |
| L-leucine⁴][γ-hydroxy-N- | | | |
| methyl-L-leucine⁹]cyclosporin A | | | |
| tocopherol acetate | 0.1 | 0.1 | 0.1 |
| salicylic acid | 0.3 | 0.3 | 0.3 |
| L-menthol | 0.3 | 0.3 | 0.3 |
| Tween 20 | 0.5 | 0.5 | 0.5 |
| perfume | typical | typical | typical |
| colorant | typical | typical | typical |
| water | balance | balance | balance |

(unit: weight%)

Formulation 1-4:

Preparation of hair tonic containing [L-threonine²][γ-hydroxy-N-methyl-L-leucine⁴][L-leucine⁵][D-2-hydroxyisovaleric acid⁸][L-leucine¹⁰] cyclosporin A

Individual ingredients were mixed and stirred, and the mixtures were completely dissolved to prepare three hair growth promoting tonics, with compositions as shown in Table 4 below. It was found that the composition 1 of Table 4 has a hair growth promoting effect at a level similar to a conventional hair tonic containing 0.1% cyclosporin A, as evaluated in an animal experiment according to Test Example 1 described later.

TABLE 4
Formulation of hair tonic

| Ingredients | Comp. 1 | Comp. 2 | Comp. 3 |
|---|---|---|---|
| ethanol | 40.0 | 40.0 | 40.0 |
| [L-threonine²][γ- | 0.1 | 1.0 | 8.0 |
| hydroxy-N-methyl-L-leucine⁴] | | | |
| [L-leucine⁵][D-2- | | | |
| hydroxyisovaleric acid⁸] | | | |
| [L-leucine¹⁰] | | | |
| cyclosporin A | | | |
| tocopherol acetate | 0.1 | 0.1 | 0.1 |
| salicylic acid | 0.3 | 0.3 | 0.3 |
| L-menthol | 0.3 | 0.3 | 0.3 |
| Tween 20 | 0.5 | 0.5 | 0.5 |
| perfume | typical | typical | typical |
| colorant | typical | typical | typical |
| water | balance | balance | balance |

(unit: weight%)

Formulation 2-1:

Preparation of hair cream containing [N-methyl-D-alanine³][γ-hydroxy-N-methyl-L-leucine⁴] cyclosporin A

Individual oil-phase and water-phase ingredients were mixed in a separate 5 container, and each mixture was completely dissolved by heating to 80° C. Two phases of the ingredients were mixed, emulsified, and cooled to room temperature. Additives such as perfume and colorant were admixed to prepare three hair creams, with compositions as shown in Table 5 below. Water was added to adjust to 100% the total weight including the oil-phase and water-phase ingredients.

It was found that the composition 1 of Table 5 has a hair growth promoting effect at a level similar to a conventional hair cream containing 0.1% cyclosporin A, as evaluated in an animal experiment according to Test Example 1 described later.

TABLE 5
Formulation of hair cream

| Ingredients | Comp. 1 | Comp. 2 | Comp. 3 |
|---|---|---|---|
| paraffin | 5.0 | 5.0 | 5.0 |
| cetostearyl alcohol | 5.5 | 5.5 | 5.5 |
| petrolatum | 5.5 | 5.5 | 5.5 |
| glycerin monostearate | 3.0 | 3.0 | 3.0 |
| polyoxyethyleneoctyl- | 3.0 | 3.0 | 3.0 |
| dodecylether | | | |
| propylparaben | 0.3 | 0.3 | 0.3 |
| [N-methyl-D-alanine³] | 0.1 | 1.0 | 8.0 |
| [γ-hydroxy-N- | | | |
| methyl-L-leucine⁴] | | | |
| cyclosporin A | | | |
| glycerin | 7.0 | 7.0 | 7.0 |
| dipropyleneglycol | 20.0 | 20.0 | 20.0 |
| polyethyleneglycol | 5.0 | 5.0 | 5.0 |
| water | balance not including perfume and colorant | | |
| perfume | typical | typical | typical |
| colorant | typical | typical | typical |

(unit: weight%)

Formulation 2-2:

Preparation of hair cream containing [γ-hydroxy-N-methyl-L-leucine⁴][alanine thiomide⁷, ⁷ψ⁸ CS—NH] cyclosporin A

Individual oil-phase and water-phase ingredients were mixed in a separate container, and each mixture was completely dissolved by heating to 80° C. Two phases of the ingredients were mixed, emulsified, and cooled to room temperature. Additives such as perfume and colorant were admixed to prepare three hair creams, with compositions as shown in Table 6 below. Water was added to adjust to 100% the total weight including the oil-phase and water-phase ingredients.

It was found that the composition 1 of Table 6 has a hair growth promoting effect at a level similar to a conventional hair cream containing 0.1% cyclosporin A, as evaluated in an animal experiment according to Test Example 1 described later.

TABLE 6
Formulation of hair cream

| Ingredients | Comp. 1 | Comp. 2 | Comp. 3 |
|---|---|---|---|
| paraffin | 5.0 | 5.0 | 5.0 |
| cetostearyl alcohol | 5.5 | 5.5 | 5.5 |
| petrolatum | 5.5 | 5.5 | 5.5 |
| glycerin monostearate | 3.0 | 3.0 | 3.0 |
| polyoxyethyleneoctyl- | 3.0 | 3.0 | 3.0 |
| dodecylether | | | |
| propylparaben | 0.3 | 0.3 | 0.3 |
| [γ-hydroxy-N-methyl-L- | 0.1 | 1.0 | 8.0 |
| leucine⁴][alanine | | | |
| thiomide⁷,⁷Ψ⁸CS—NH] | | | |
| cyclosporin A | | | |
| glycerin | 7.0 | 7.0 | 7.0 |
| dipropyleneglycol | 20.0 | 20.0 | 20.0 |
| polyethyleneglycol | 5.0 | 5.0 | 5.0 |
| water | balance not including perfume and colorant | | |
| perfume | typical | typical | typical |
| colorant | typical | typical | typical |

(unit: weight%)

Formulation 2-3:

Preparation of hair cream containing [γ-hydroxy-N-methyl-L-leucine⁴][γ-hydroxy-N-methyl-L-leucine⁹] cyclosporin A

Individual oil-phase and water-phase ingredients were mixed in a separate container, and each mixture was completely dissolved by heating to 80° C. Two phases of the ingredients were mixed, emulsified, and cooled to room temperature. Additives such as perfume and colorant were admixed to prepare three hair creams, with compositions as shown in Table 7 below. Water was added to adjust to 100% the total weight including the oil-phase and water-phase ingredients.

It was found that the composition 1 of Table 7 has a hair growth promoting effect at a level similar to a conventional hair cream containing 0.1% cyclosporin A, as evaluated in an animal experiment according to Test Example 1 described later

TABLE 7
Formulation of hair cream

| Ingredients | Comp. 1 | Comp. 2 | Comp. 3 |
|---|---|---|---|
| paraffin | 5.0 | 5.0 | 5.0 |
| cetostearyl alcohol | 5.5 | 5.5 | 5.5 |
| petrolatum | 5.5 | 5.5 | 5.5 |
| glycerin monostearate | 3.0 | 3.0 | 3.0 |
| polyoxyethyleneoctyldo- | 3.0 | 3.0 | 3.0 |
| decylether | | | |
| propylparaben | 0.3 | 0.3 | 0.3 |
| [γ-hydroxy-N-methyl-L- | 0.1 | 1.0 | 8.0 |
| leucine⁴][γ-hydroxy-N- | | | |
| methyl-L-leucine⁹] | | | |
| cyclosporin A | | | |
| glycerin | 7.0 | 7.0 | 7.0 |
| dipropyleneglycol | 20.0 | 20.0 | 20.0 |
| polyethyleneglycol | 5.0 | 5.0 | 5.0 |
| water | balance not including perfume and colorant | | |
| perfume | typical | typical | typical |
| colorant | typical | typical | typical |

(unit: weight%)

Formulation 2-4:

Preparation of hair cream containing [L-threonine²][γ-hydroxy-N-methyl-L-leucine⁴][L-leucine⁵][D-2-hydroxyisovaleric acid⁸][L-leucine¹⁰] cyclosporin A

Individual oil-phase and water-phase ingredients were mixed in a separate container, and each mixture was completely dissolved by heating to 80° C. Two phases of the ingredients were mixed, emulsified, and cooled to room temperature. Additives such as perfume and colorant were admixed to prepare three hair creams, with compositions as shown in Table 8 below. Water was added to adjust to 100% the total weight including the oil-phase and water-phase ingredients.

It was found that the composition 1 of Table 8 has a hair growth promoting effect at a level similar to a conventional hair cream containing 0.1% cyclosporin A, as evaluated in an animal experiment according to Test Example 1 described later.

TABLE 8
Formulation of hair cream

| Ingredients | Comp. 1 | Comp. 2 | Comp. 3 |
|---|---|---|---|
| paraffin | 5.0 | 5.0 | 5.0 |
| cetostearyl alcohol | 5.5 | 5.5 | 5.5 |
| petrolatum | 5.5 | 5.5 | 5.5 |
| glycerin monostearate | 3.0 | 3.0 | 3.0 |
| polyoxyethyleneoctyl- | 3.0 | 3.0 | 3.0 |
| dodecylether | | | |
| propylparaben | 0.3 | 0.3 | 0.3 |
| [L-threonine²][γ- | 0.1 | 1.0 | 8.0 |
| hydroxy-N-methyl-L- | | | |
| leucine⁴][L-leucine⁵] | | | |
| [D-2-hydroxyisovaleric- | | | |
| acid⁸][L-leucine¹⁰] | | | |
| cyclosporin A | | | |
| glycerin | 7.0 | 7.0 | 7.0 |
| dipropyleneglycol | 20.0 | 20.0 | 20.0 |
| polyethyleneglycol | 5.0 | 5.0 | 5.0 |
| water | balance not including perfume and colorant | | |
| perfume | typical | typical | typical |
| colorant | typical | typical | typical |

(unit: weight%)

Formulation 3-1:

Preparation of shampoo containing [N-methyl-D-alanine³][γ-hydroxy-N-methyl-L-leucine⁴] cyclosporin A

All individual ingredients, except perfume, colorant and water, were mixed and the mixture was completely dissolved by heating, while stirring. After cooling to room temperature, the mixture was mixed with perfume and colorant. Water was finally added to adjust to 100% the total weight, to prepare three shampoos, with compositions as shown in Table 9 below.

TABLE 9
Formulation of shampoo

| Ingredients | Comp. 1 | Comp. 2 | Comp. 3 |
|---|---|---|---|
| sodium POE | 40.0 | 40.0 | 40.0 |
| laurylsulfuric acid | | | |
| (30 wt % aqueous solution) | | | |
| palm oil fattyacid | 3.0 | 3.0 | 3.0 |
| diethanolamide | | | |
| propyleneglycol | 2.0 | 2.0 | 2.0 |
| methyl paraoxybenzoic acid | 0.2 | 0.2 | 0.2 |
| ehtanol | 2.0 | 2.0 | 2.0 |
| [N-methyl-D-alanine³] | 1.0 | 3.0 | 10.0 |
| [γ-hydroxy-N-methyl- | | | |
| L-leucine⁴]cyclosporin A | | | |
| salicylic acid | 0.3 | 0.3 | 0.3 |
| L-menthol | 0.3 | 0.3 | 0.3 |
| perfume | typical | typical | typical |
| colorant | typical | typical | typical |
| water | balance | balance | balance |

(unit: weight%)

Formulation 3-2:

Preparation of shampoo containing [γ-hydroxy-N-methyl-L-leucine⁴][alanine thiomide⁷, ⁷ψ⁸ CS—NH] cyclosporin A

All individual ingredients, except perfume, colorant and water, were mixed and the mixture was completely dissolved by heating, while stirring. After cooling to room temperature, the mixture was mixed with perfume and colorant. Water was finally added to adjust to 100% the total weight, to prepare three shampoos, with compositions as shown in Table 10 below.

TABLE 10
Formulation of shampoo

| Ingredients | Comp. 1 | Comp. 2 | Comp. 3 |
|---|---|---|---|
| sodium POE | 40.0 | 40.0 | 40.0 |
| laurylsulfuric acid | | | |
| (30 wt % aqueous solution) | | | |
| palm oil fattyacid | 3.0 | 3.0 | 3.0 |
| diethanolamide | | | |
| propyleneglycol | 2.0 | 2.0 | 2.0 |
| methyl paraoxybenzoic acid | 0.2 | 0.2 | 0.2 |
| ehtanol | 2.0 | 2.0 | 2.0 |
| [γ-hydroxy-N-methyl- | 1.0 | 3.0 | 10.0 |
| L-leucine⁴] [alanine | | | |
| thiomide⁷,⁷Ψ⁸CS—NH] | | | |
| cyclosporin A | | | |
| salicylic acid | 0.3 | 0.3 | 0.3 |
| L-menthol | 0.3 | 0.3 | 0.3 |
| perfume | typical | typical | typical |
| colorant | typical | typical | typical |
| water | balance | balance | balance |

Formulation 3-3:

Preparation of shampoo, containing [γ-hydroxy-N-methyl-L-leucine⁴][γ-hydroxy-N-methyl-L-leucine⁹] cyclosporin A

All individual ingredients, except perfume, colorant and water, were mixed and the mixture was completely dissolved by heating, while stirring. After cooling to room temperature, the mixture was mixed with perfume and colorant. Water was finally added to adjust to 100% the total weight, to prepare three shampoos, with compositions as shown in Table 11 below.

TABLE 11
Formulation of shampoo

| | (unit: weight %) | | |
|---|---|---|---|
| Ingredients | Comp. 1 | Comp. 2 | Comp. 3 |
| sodium POE laurylsulfuric acid | 40.0 | 40.0 | 40.0 |
| (30 wt % aqueous solution) | | | |
| palm oil fattyacid diethanolamide | 3.0 | 3.0 | 3.0 |
| propyleneglycol | 2.0 | 2.0 | 2.0 |
| methyl paraoxybenzoic acid | 0.2 | 0.2 | 0.2 |
| ehtanol | 2.0 | 2.0 | 2.0 |
| [γ-hydroxy-N-methyl-L-leucine⁴] | 1.0 | 3.0 | 10.0 |
| [γ-hydroxy-N-methyl-L-leucine⁹] | | | |
| cyclosporin A | | | |
| salicylic acid | 0.3 | 0.3 | 0.3 |
| L-menthol | 0.3 | 0.3 | 0.3 |
| perfume | typical | typical | typical |
| colorant | typical | typical | typical |
| water | balance | balance | balance |

Formulation 3-4:

Preparation of shampoo containing [L-threonine²][γ-hydroxy-N-methyl-L-leucine⁴][L-leucine⁵][D-2-hydroxyisovaleric acid⁸][L-leucine¹⁰] cyclosporin A

All individual ingredients, except perfume, colorant and water, were mixed and the mixture was completely dissolved by heating, while stirring. After cooling to room temperature, the mixture was mixed with perfume and colorant. Water was finally added to adjust to 100% the total weight, to prepare three shampoos, with compositions as shown in Table 12 below.

TABLE 12
Formulation of shampoo

| | (unit: weight %) | | |
|---|---|---|---|
| Ingredients | Comp. 1 | Comp. 2 | Comp. 3 |
| sodium POE laurylsulfuric acid | 40.0 | 40.0 | 40.0 |
| (30 wt % aqueous solution) | | | |
| palm oil fattyacid diethanolamide | 3.0 | 3.0 | 3.0 |
| propyleneglycol | 2.0 | 2.0 | 2.0 |
| methyl paraoxybenzoic acid | 0.2 | 0.2 | 0.2 |
| ehtanol | 2.0 | 2.0 | 2.0 |
| [L-threonine²][γ-hydroxy- | 1.0 | 3.0 | 10.0 |
| N-methyl-L-leucine⁴][L- | | | |
| leucine⁵][D-2- | | | |
| hydroxyisovaleric acid⁸][L- | | | |
| leucine¹⁰] | | | |
| cyclosporin A | | | |
| salicylic acid | 0.3 | 0.3 | 0.3 |
| L-menthol | 0.3 | 0.3 | 0.3 |
| perfume | typical | typical | typical |
| colorant | typical | typical | typical |
| water | balance | balance | balance |

Formulation 4-1:

Preparation of hair conditioner containing [N-methyl-D-alanine³][γ-hydroxy-N-methyl-L-leucine⁴] cyclosporin A

Individual oil-phase and water-phase ingredients were mixed in a separate container, and each mixture was completely dissolved by heating to 80° C. Two phases of the ingredients were mixed, emulsified, and cooled to room temperature. Additives such as perfume and colorant were admixed to prepare three hair conditioners, with compositions as shown in Table 13 below. Water was added to adjust to 100% the total weight including the oil-phase and water-phase ingredients.

TABLE 13
Formulation of hair conditioner

| | (unit: weight %) | | |
|---|---|---|---|
| Ingredients | Comp. 1 | Comp. 2 | Comp. 3 |
| cetanol | 3.0 | 3.0 | 3.0 |
| self-emulsifiableglycerol- | 2.0 | 2.0 | 3.0 |
| monostearate | | | |
| squalene | 10.0 | 10.0 | 10.0 |
| [N-methyl-D-alanine³][γ- | 1.0 | 5.0 | 10.0 |
| hydroxy-N-methyl-L- | | | |
| leucine⁴]cyclosporin A | | | |
| propyleneglycol | 2.0 | 2.0 | 2.0 |
| stearyldimethyl benzylammonium | 8.0 | 8.0 | 8.0 |
| chloride | | | |
| (25 wt % aqueous solution) | | | |
| methyl paraoxybenzoic acid | 0.2 | 0.2 | 0.2 |
| salicylic acid | 0.3 | 0.3 | 0.3 |
| L-menthol | 0.3 | 0.3 | 0.3 |
| water | balance | balance | balance |
| perfume | typical | typical | typical |
| colorant | typical | typical | typical |

Formulation 4-2:

Preparation of hair conditioner containing [γ-hydroxy-N-methyl-L-leucine⁴][alanine thiomide⁷, ⁷ψ⁸ CS—NH] cyclosporin A

Individual oil-phase and water-phase ingredients were mixed in a separate container, and each mixture was completely dissolved by heating to 80° C. Two phases of the ingredients were mixed, emulsified, and cooled to room temperature. Additives such as perfume and colorant were admixed to prepare three hair conditioners, with compositions as shown in Table 14 below. Water was added to adjust to 100% the total weight including the oil-phase and water-phase ingredients.

TABLE 14
Formulation of hair conditioner

| | (unit: weight %) | | |
|---|---|---|---|
| Ingredients | Comp. 1 | Comp. 2 | Comp. 3 |
| cetanol | 3.0 | 3.0 | 3.0 |
| self-emulsifiable glycerol- | 2.0 | 2.0 | 3.0 |
| monostearate | | | |
| squalene | 10.0 | 10.0 | 10.0 |
| [γ-hydroxy-N-methyl-L- | 1.0 | 5.0 | 10.0 |
| leucine⁴] [alanine thiomide⁷, ⁷ψ⁸CS- | | | |
| NH] cyclosporin A | | | |
| propyleneglycol | 2.0 | 2.0 | 2.0 |
| stearyldimethyl benzylammonium | 8.0 | 8.0 | 8.0 |
| chloride | | | |
| (25 wt % aqueous solution) | | | |
| methyl paraoxybenzoic acid | 0.2 | 0.2 | 0.2 |
| salicylic acid | 0.3 | 0.3 | 0.3 |
| L-menthol | 0.3 | 0.3 | 0.3 |
| water | balance | balance | balance |
| perfume | typical | typical | typical |
| colorant | typical | typical | typical |

Formulation 4-3:

Preparation of hair conditioner containing [γ-hydroxy-N-methyl-L-leucine⁴][γ-hydroxy-N-methyl-L-leucine⁹] cyclosporin A

Individual oil-phase and water-phase ingredients were mixed in a separate container, and each mixture was completely dissolved by heating to 80° C. Two phases of the ingredients were mixed, emulsified, and cooled to room temperature. Additives such as perfume and colorant were admixed to prepare three hair conditioners, with compositions as shown in Table 15 below. Water was added to adjust to 100% the total weight including the oil-phase and water-phase ingredients.

TABLE 15
Formulation of hair conditioner

| | (unit: weight %) | | |
|---|---|---|---|
| Ingredients | Comp. 1 | Comp. 2 | Comp. 3 |
| cetanol | 3.0 | 3.0 | 3.0 |
| self-emulsifiable glycerol- | 2.0 | 2.0 | 3.0 |
| monostearate | | | |
| squalene | 10.0 | 10.0 | 10.0 |
| [γ-hydroxy-N-methyl-L- | 1.0 | 5.0 | 10.0 |
| leucine⁴][γ-hydroxy-N-methyl- | | | |
| L-leucine⁹] cyclosporin A | | | |
| propyleneglycol | 2.0 | 2.0 | 2.0 |
| stearyldimethyl benzylammonium | 8.0 | 8.0 | 8.0 |
| chloride | | | |
| (25 wt % aqueous solution) | | | |
| methyl paraoxybenzoic acid | 0.2 | 0.2 | 0.2 |
| salicylic acid | 0.3 | 0.3 | 0.3 |
| L-menthol | 0.3 | 0.3 | 0.3 |
| water | balance | balance | balance |
| perfume | typical | typical | typical |
| colorant | typical | typical | typical |

Formulation 4-4:

Preparation of hair conditioner containing [L-threonine²][γ-hydroxy-N-methyl-L-leucine⁴][L-leucine⁵][D-2-hydroxyisovaleric acid⁸][L-leucine¹⁰] cyclosporin A

Individual oil-phase and water-phase ingredients were mixed in a separate container, and each mixture was completely dissolved by heating to 80° C. Two phases of the ingredients were mixed, emulsified, and cooled to room temperature. Additives such as perfume and colorant were admixed to prepare three hair conditioners, with compositions as shown in Table 16 below. Water was added to adjust to 100% the total weight including the oil-phase and water-phase ingredients.

TABLE 16
Formulation of hair conditioner

| | (unit: weight %) | | |
|---|---|---|---|
| Ingredients | Comp. 1 | Comp. 2 | Comp. 3 |
| cetanol | 3.0 | 3.0 | 3.0 |
| self-emulsifiable glycerol- | 2.0 | 2.0 | 3.0 |
| monostearate | | | |
| squalene | 10.0 | 10.0 | 10.0 |
| [L-threonine²][γ-hydroxy-N- | 1.0 | 5.0 | 10.0 |
| methyl-L-leucine⁴][L- | | | |
| leucine⁵][D-2-hydroxyisovaleric | | | |
| acid⁸][L-leucine¹⁰] | | | |
| cyclosporin A | | | |
| propyleneglycol | 2.0 | 2.0 | 2.0 |
| stearyldimethyl benzylammonium | 8.0 | 8.0 | 8.0 |
| chloride | | | |
| (25 wt % aqueous solution) | | | |
| methyl paraoxybenzoic acid | 0.2 | 0.2 | 0.2 |
| salicylic acid | 0.3 | 0.3 | 0.3 |
| L-menthol | 0.3 | 0.3 | 0.3 |
| water | balance | balance | balance |
| perfume | typical | typical | typical |
| colorant | typical | typical | typical |

### TEST EXAMPLE 1

### Evaluation of Hair Restoring Effect in Mice

Female C57BL/6 mice of ages 6 to 7 weeks were utilized. After removing hairs on the middle of the back with an electric shaver, the mice were weighed and randomly assigned to the test groups with an even distribution of weights. After one day of adaptation, the mice were applied with cyclosporin A and the cyclosporin A derivatives prepared by HPLC in Examples 1 to 4 to their hair removed areas once a day at a dose of 100 μl (conc. 0.1% w/v), for 30 days. The results were determined by visual examination, in terms of degrees of hair regrowth. With respect to respective hair-removed areas, rates of new hair growth were examined and compared.

As can be seen in Table 17, cyclosporin derivatives of the invention have a significant hair growth promoting effect, compared to the control in which mice were applied with a vehicle only. Further, the derivatives show a similar level of hair growth promoting effect, with respect to cyclosporin A. Meanwhile, over the course of 30 days, as comparing the appearance of the backs, the mice of the control and all test groups showed no specific skin irritation.

TABLE 17
Hair restore in mice

| | Area rate |
|---|---|
| | of hair |
| Compound applied | regrowth (%) |
| vehicle | 35 |
| cyclosporin A | 91 |
| [N-methyl-D-alanine³][γ- | 95 |
| hydroxy-N-methyl-L-leucine⁴] | |
| cyclosporin A | |
| [γ-hydroxy-N-methyl-L-leucine⁴] | 91 |
| [alanine thiomide⁷, ⁷ψ⁸ CS-NH] cyclosporin A | |
| [γ-hydroxy-N-methyl-L-leucine⁴][γ- | 85 |
| hydroxy-N-methyl-L-leucine⁹] cyclosporin A | |
| [L-threonine²][γ-hydroxy-N-methyl-L- | 90 |
| leucine⁴][L-leucine⁵][D-2-hydroxyisovaleric | |
| acid⁸][L-leucine¹⁰] cyclosporin A | |

### TEST EXAMPLE 2

### Evaluation of Non-Immunosuppressive Activity In Vitro

To compare immunosuppressive activities of cyclosporin derivatives according to the invention with that of cyclosporin A, the MLR method (Mixed Lymphocyte Reaction method, J. Antibiotics, 1994, 47:208-215), employing splenocytes obtained from two distinct species of mice, was used.

Responder cells from BALB/c mice were mixed with an equal number of stimulator cells from C57BL/6 mice pre-treated with mitomycin. The mixed splenocytes were respectively treated with cyclosporin A and the cyclosporin derivatives of the invention, and incubated in RPMI media (supplemented with mercaptoethanol and 10% fetal bovine serum) for 4 days. After 4 days of incubation, ³H-thymidine was added to the mixtures and incubated for an additional 4 hours. Then, the amount of thymidine incorporated into the cells was measured (liquid scintillation counter), and IC₅₀ (μg/ml) of respective cyclosporins were calculated.

As a result, IC₅₀ (μg/ml) of cyclosporin A was 0.022, 0.042 and 0.040 in triplicate cultures, respectively. On the other hand, the IC₅₀ (μg/ml) values for [N-methyl-D-alanine³][γ-hydroxy-N-methyl-L-leucine⁴] cyclosporin A were 8.2, 8.8 and 9.3, for [γ-hydroxy-N-methyl-L-leucine⁴][alanine thiomide⁷, ⁷ψ⁸ CS—NH] cyclosporin A, 10.1, 13.2 and 13.9, for γ-hydroxy-N-methyl-L-leucine⁴][, γ-hydroxy-N-methyl-L-leucine⁹] cyclosporin A, 18.2, 19.2 and 17.2, and for [L-threonine²][γ-hydroxy-N-methyl-L-leucine⁴][L-leucine⁵][D-2-hydroxyisovaleric acid⁸][L-leucine¹⁰] cyclosporin A, 30.1, 29.0 and 19.2, indicating more than 100-fold decrease in its immunosuppressive activity, compared to cyclosporin A.

These findings prove that [γ-hydroxy-N-methyl-L-leucine⁴] cyclosporin derivatives which are added with a hydroxyl group by microbial metabolism to gamma carbon in N-methyl-L-leucine, the residue No. 4 of cyclosporin, have very weak immunosuppressive activities, compared to unmodified cyclosporin A, while they retain an excellent hair growth effect.

On the basis of the foregoing results, the cyclosporin derivatives of the invention may be formulated in any form including liquid formulations, sprays, gels, pastes, emulsions, creams, conditioners, shampoos, and the like. A variety of forms are available though, considering their high commercial demand, hair tonics, creams, conditioners, and shampoos are provided herein. As revealed in the above the Test Example 1, the cyclosporin derivatives exhibit an excellent hair growth promoting effect, compared to the control.

### INDUSTRIAL APPLICABILITY

As apparent from the above description, the present invention provides a hair growth promoting agent, comprising a cyclosporin derivative as an active ingredient, exhibits an excellent hair growth effect, while it shows a very weak immunosuppressive activity, compared to unmodified cyclosporin A.

1. A hair growth promoting agent comprising a [γ-hydroxy-N-methyl-L-leucine⁴] cyclosporin derivative represented by Chemical Formula 1 below, having an excellent hair restoring effect with non-immunosuppressive activity, as an active ingredient: 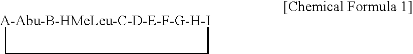
in which
A represents N-methyl-(4R)-4-[(E)-2-butenyl]-4-methyl-L-threonine (MeBmt), (2S,3R,4R,6E)-3-sulfhydryl-4-methyl-2-(methylamino)-6-octenoic acid or (2S,4R,6E)-3-oxo-4-methyl-2-(methylamino)-6-octenoic acid;
Abu represents L-γ-aminobutyric acid (Abu);
B represents a D-amino acid represented by the general formula 1,
CH₃NH—CH(R)—COOH [General formula 1]
in which,
R is one selected from the group consisting of hydrogen, C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino, and X—R′ represented by the general formula 2 below,
—X—R′ [General formula 2]
in which,
X is oxygen or sulfur, and
R′ is one selected from the group consisting of hydrogen, and C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino;
C represents L-valine or L-norvaline;
D represents N-methyl-L-leucine, γ-hydroxy-N-methyl-L-leucine or L-leucine;
E represents L-alanine or L-alanine thioamide ([⁷ψ⁸ CS—NH], NH—CHCH₃—CS—);
F represents D-2-hydroxyisovaleric acid or a D-amino acid represented by the general formula 3,
—NH—CH(CH₂R)—COOH [General formula 3]
in which,
R is hydrogen or X—R′ represented by the general formula 4,
—X—R′ [General formula 4]
in which,
X is oxygen or sulfur, and
R′ is one selected from the group consisting of hydrogen, and C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino;
G represents N-methyl-L-leucine, γ-hydroxy-N-methyl-L-leucine or L-leucine;
H represents N-methyl-L-leucine, γ-hydroxy-N-methyl-L-leucine or L-leucine; and
I represents N-methyl-L-valine or L-valine,
excepted that if B is sarcosine, then C is L-valine, D is N-methyl-L-leucine, E is L-alanine, F is D-alanine, G is N-methyl-L-leucine, H is N-methyl-L-leucine, and I is N-methyl-L-valine.

2. The hair growth promoting agent as set forth in claim 1, wherein the [γ-hydroxy-N-methyl-L-leucine⁴] cyclosporin derivative is represented by Chemical Formula 2: 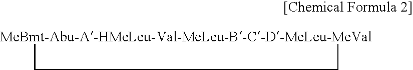
in which
MeBmt represents N-methyl-(4R)-4-[(E)-2-butenyl]-4-methyl-L-threonine;
Abu represents L-α-aminobutyric acid (Abu);
A′ represents N-methyl-D-alanine, D-2-(methylamino)pent-4-enoyl, N-methyl-D-aminobutyric acid, N-methyl-D-norvaline, D-2-(methylamino)hexa-4-ynoyl, D-2-(methylamino)pent-4-ynoyl, D-2-methylthio-sarcosine, N-methyl-O-propenyl-D-serine, N-methyl-D-serine or sarcosine;
HMeLeu represents γ-hydroxy-N-methyl-L-leucine;
Val represents L-valine;
MeLeu represents N-methyl-L-leucine;
B′ represents L-alanine or L-alanine thioamide ([⁷ψ⁸ CS—NH], NH—CHCH₃—CS—);
C′ represents D-2-hydroxyisovaleric acid, or a D-amino acid represented by the general formula 3,
—NH—CH(CH₂R)—COOH [General formula 3]
in which,
R is hydrogen or X—R′ represented by the general formula 4,
—X—R′ [General formula 4]
in which,
X is oxygen or sulfur, and
R′ is one selected from the group consisting of hydrogen, and C₁-C₆ straight or branched alkyl, alkenyl or alkynyl moieties, substituted or unsubstituted with one or more selected from the group consisting of amino, hydroxy, halo, haloalkyl, ester, alkoxy, cyano, nitro, alkylamino, and dialkylamino;
D′ represents N-methyl-L-leucine, γ-hydroxy-N-methyl-L-leucine or L-leucine; and
MeVal represents N-methyl-L-valine,
excepted that if A is sarcosine, then B is L-alanine, C is D-alanine, and D is N-methyl-L-leucine.

3. The hair growth promoting agent as set forth in claim 1, wherein the [γ-hydroxy-N-methyl-L-leucine⁴] cyclosporin derivative is represented by Chemical Formula 3: 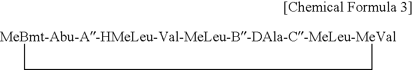
in which
MeBmt represents N-methyl-(4R)-4-[(E)-2-butenyl]-4-methyl-L-threonine;
Abu represents L-α-aminobutyric acid (Abu);
A″ represents N-methyl-D-alanine, D-2-(methylamino)pent-4-enoyl, N-methyl-D-aminobutyric acid, N-methyl-D-norvaline, D-2-(methylamino)hexa-4-ynoyl, D-2-(methylamino)pent-4-ynoyl, D-2-methylthio-sarcosine, N-methyl-O-propenyl-D-serine, N-methyl-D-serine or sarcosine;
HMeLeu represents γ-hydroxy-N-methyl-L-leucine;
Val represents L-valine;
MeLeu represents N-methyl-L-leucine;
B″ represents L-alanine or L-alanine thioamide ([⁷ψ⁸ CS—NH], NH—CHCH₃—CS—);
DAla represents D-alanine;
C″ represents N-methyl-L-leucine, γ-hydroxy-N-methyl-L-leucine or L-leucine; and
MeVal represents N-methyl-L-valine,
excepted that if A is sarcosine, then B is L-alanine, and C is N-methyl-L-leucine.

4. The hair growth promoting agent as set forth in claim 3, comprising [N-methyl-D-alanine³][γ-hydroxy-N-methyl-L-leucine⁴] cyclosporin A as an active ingredient.

5. The hair growth promoting agent as set forth in claim 3, comprising [D-2-(methylamino)pent-4-enoyl³][γ-hydroxy-N-methyl-L-leucine⁴] cyclosporin A as an active ingredient.

6. The hair growth promoting agent as set forth in claim 3, comprising [N-methyl-D-Abu³][γ-hydroxy-N-methyl-L-leucine⁴] cyclosporin A as an active ingredient.

7. The hair growth promoting agent as set forth in claim 3, comprising [N-methyl-D-Norvaline³][γ-hydroxy-N-methyl-L-leucine⁴] cyclosporin A as an active ingredient.

8. The hair growth promoting agent as set forth in claim 3, comprising [D-2-(methylamino)hexa-4-ynoyl³][γ-hydroxy-N-methyl-L-leucine⁴] cyclosporin A as an active ingredient.

9. The hair growth promoting agent as set forth in claim 3, comprising [D-2-(methylamino)pent-4-ynoyl³][γ-hydroxy-N-methyl-L-leucine⁴] cyclosporin A as an active ingredient.

10. The hair growth promoting agent as set forth in claim 3, comprising [D-2-methylamino-Sar³][γ-hydroxy-N-methyl-L-leucine⁴] cyclosporin A as an active ingredient.

11. The hair growth promoting agent as set forth in claim 3, comprising [N-methyl-O-propenyl-D-serine ³][γ-hydroxy-N-methyl-L-leucine⁴] cyclosporin A as an active ingredient.

12. The hair growth promoting agent as set forth in claim 3, comprising [N-methyl-D-serine³][γ-hydroxy-N-methyl-L-leucine⁴] cyclosporin A as an active ingredient.

13. The hair growth promoting agent as set forth in claim 3, comprising [γ-hydroxy-N-methyl-L-leucine⁴][alanine thioamide⁷, ⁷ψ⁸ CS—NH] cyclosporin A as an active ingredient.

14. The hair growth promoting agent as set forth in claim 3, comprising [γ-hydroxy-N-methyl-L-leucine⁴][γ-hydroxy-N-methyl-L-leucine⁹] cyclosporin A as an active ingredient.

15. The hair growth promoting agent-as set forth in claim 3, comprising [γ-hydroxy-N-methyl-L-leucine⁴][D-serine⁸] cyclosporin A as an active ingredient.

16. The hair growth promoting agent as set forth in claim 3, comprising [L-threonine²][γ-hydroxy-N-methyl-L-leucine ⁴][L-leucine⁵][D-2-hydroxyisovaleric acid⁸][L-leucine¹⁰] cyclosporin A as an active ingredient.

17. The hair growth promoting agent as set forth in any one of claims 1 to 16, which is formulated in a form selected from the group consisting of liquid formulation, spray, gel, paste, emulsion, cream, conditioner and shampoo.
